(19) United States
(12) Patent Application Publication
(10) Pub. No.: US 2024/0002768 A1
(43) Pub. Date: Jan. 4, 2024

(54) MODULAR FIXED-BED BIOREACTOR SYSTEMS AND METHODS OF USING THE SAME

(71) Applicant: CORNING INCORPORATED, Corning, NY (US)

(72) Inventor: Paul Kevin Seeto, Clinton, MA (US)

(21) Appl. No.: 18/038,855

(22) PCT Filed: Nov. 17, 2021
(86) PCT No.: PCT/US2021/059706

§ 371 Date: May 25, 2023

Related U.S. Application Data
(60) Provisional application No. 63/118,067, filed on Nov. 25, 2020.

(51) Int. Cl. C12M 1/12; C12M 1/00

(52) U.S. Cl. CPC C12M 25/18; C12M 25/14; C12M 29/14

(57) ABSTRACT

A fixed-bed bioreactor system for culturing cells is provided. The system includes a plurality of cell culture subunits, each cell culture subunit including a distribution plate with a major surface to support a cell culture substrate, an inlet, and a plurality of outlets disposed on the major surface and in fluid communication with the inlet. The subunit also includes a cell culture substrate disposed on the major surface of the distribution plate. The system further includes a plurality of input lines for supplying at least one of cells, cell culture media, nutrients, and reagents to the plurality of cell culture subunits, each input line of the plurality of input lines being fluidly connected to the inlet. The plurality of outlets is configured to distribute at least one of cells, cell culture media, nutrients, and reagents from the plurality of input lines substantially uniformly across the cell culture substrate.

### CROSS-REFERENCE TO RELATED APPLICATIONS

This application claims the benefit of priority under 35 U.S.C. § 119 of U.S. Provisional Application Ser. No. 63/118,067 filed on Nov. 25, 2020, the content of which is relied upon and incorporated herein by reference in its entirety.

### FIELD OF THE DISCLOSURE

This disclosure general relates to substrates for culturing cells, as well as systems and methods for culturing cells. In particular, the present disclosure relates to cell culturing substrates, bioreactor systems incorporating such substrates, and methods of culturing cells using such substrates, including modular and scalable substrates, vessels, and systems.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells. In addition, traditional in-vitro cell culture on 2D culture substrates fails to simulate an in-vivo environment. Since almost all cells in the in-vivo environment are surrounded by other cells and extracellular matrix (ECM) in a three-dimensional (3D) fashion, 2D cell culture does not adequately simulate the natural 3D environment of cells. Cells in 2D culture are forced to adhere to a rigid surface and are geometrically constrained, adopting a flat morphology which alters the cytoskeleton regulation that is important in intracellular signaling, and consequently can affect cell growth, migration, and apoptosis. Moreover, organization of the ECM, which is significant to cell differentiation, proliferation, and gene expression, is absent in most 2D cells. These limitations of 2D cultures often result in biological responses in-vitro that are strikingly different from what is observed in-vivo.

Currently, in drug discovery, the standard procedure of screening compounds starts with 2D cell culture-based tests, followed by animal model tests, and then clinical trials. According to publicly available data, only about 10% of the compounds progress successfully through clinical development. Many drugs fail during clinical trials (especially during phase III, which is the most expensive phase of clinical development) largely due to the lack of clinical efficacy and/or unacceptable toxicity. A portion of these failures is attributed to data collected from the 2D culture tests in which the cellular response to drug(s) is altered due to their unnatural microenvironment. Due to the high costs associated with drug discovery, demand has risen for the ability to dismiss ineffective and/or unacceptable toxic compounds as early in the drug discovery process as possible. In-vitro cell-based systems that can more realistically mimic the in-vivo cell behaviors and provide more predictable results to in-vivo tests are presently being considered.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a fixed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, fixed-bed bioreactor systems that contain a fixed bed of support or matrix systems to entrap the cells have been previously disclosed U.S. Pat. Nos. 4,833,083; 5,501,971; and 5,510,262. Fixed bed matrices usually are made of porous particles as substrates or non-woven microfibers of polymer. Such bioreactors can function as recirculation flow-through bioreactors. One of the significant issues with such bioreactors is the non-uniformity of cell distribution inside the fixed bed. In effect, the fixed bed functions as depth filter with cells predominantly trapped at the inlet regions, resulting in a gradient of cell distribution during the inoculation step. In addition, due to random fiber packaging, flow resistance and cell trapping efficiency of cross sections of the fixed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of conventional fixed bed systems is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the fixed bed during cells harvesting step. It is based on loosening the fixed bed matrix and agitation or stirring of fixed bed particles to allow porous matrices to collide and thus detach the cells. However, this approach is laborious and may cause significant cells damage, thus reducing overall cell viability.

An example of a fixed-bed bioreactor currently on the market is the iCellis® by produced by Pall Corporation. The iCellis uses small strips of cell substrate material consisting of randomly oriented fibers in a non-woven arrangement. These strips are fixed into a vessel to create a fixed bed. However, as with similar solutions on the market, there are drawbacks to this type of fixed-bed substrate. Specifically, non-uniform packing of the substrate strips creates visible channels within the fixed bed, leading to preferential and non-uniform media flow and nutrient distribution through the fixed bed. Studies of the iCellis® have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the fixed bed may improve dispersion, but would have other drawbacks (i.e., “necessary agitation for better dispersion during inoculation and transfection would induce increased shear stress, in turn leading to reduced cell viability.” Id.). Another study noted of the iCellis® that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one fixed bed and another of the iCellis®, it can be difficult for customers to predict cell culture performance, since the substrate varies between cultures. Furthermore, the packed substrate of the iCellis® makes efficiently harvesting cells very difficult or impossible, as it is believed that cells are entrapped by the fixed bed.

In each of the above described technologies, a protease treatment may be used to harvest the cells. However, commonly used harvesting procedures, such as protease treatment, subject the cells to harsh conditions which may damage cell structure and function. Additionally, protease treatment alone often causes only a limited amount of cell detachment. For the fixed bed material, the problem, in part, results from the densely packed nature of the fixed bed material which makes it more difficult to circulate the protease agent throughout the bed and increase the yield of cells harvested. Similarly, it can be difficult to circulate the protease agent through interior spaces of the 3D matrix, which in turn makes it difficult to dislodge cells during the harvest process. This difficulty is compounded by the presence of extracellular macromolecules secreted by the cultured cells that serve to attach the cells to the surface of the fixed bed material or to the surface of the matrix.

Either alternatively, or in combination with the protease treatment, methods and systems for harvesting cells have been developed that apply mechanical force to release cultured cells from the fixed bed material or the 3D matrix. For example, the fixed bed material or the 3D matrix, or a larger system including the fixed bed material or the 3D matrix, may be shaken or vibrated to release the cultured cells. Application of mechanical force may also cause physical damage to the cultured cells, which in turn reduces cell culture yields.

Conventional platforms based on packed-bed bioreactors have the limitation that, when the cell density increases towards its maximum level, the cells at the rear end of the bioreactor (with respect to the flow path through the bioreactor) cannot obtain enough nutrition or oxygen, and cell productivity will thus be inhibited. This depletion of nutrients or oxygen can be viewed as a gradient of nutrient and/or oxygen supply through the flow path of the packed-bed. To reduce the development of such a nutrient/oxygen gradient that is detrimental to cell functionality, fixed beds can be designed to have relatively short media perfusion path. However, such designs significantly impact the reactor scalability in bioprocess therapeutics manufacturing. For example, while suspension stirred tank bioreactors can be scaled up to 2,000 L or to 10,000 L, typical packed bed bioreactors are only scalable up to 50 L of capacity. While manufacturing of viral vectors for early-phase clinical trials is possible with existing platforms, there is a need for a platform that can produce high-quality product in greater numbers in order to reach late-stage commercial manufacturing scale. In particular, there is a need for a platform and methods for compartmentalizing the packed bed while managing fluid flow of cells and nutrients through the bed, and reducing nutrient and/or oxygen gradients through the packed bed.

### SUMMARY

According to an embodiment of this disclosure, a fixed-bed bioreactor system for culturing cells is provided. The system includes: a plurality of cell culture subunits, each cell culture subunit including: a distribution plate with a major surface to support a cell culture substrate, an inlet, and a plurality of outlets disposed on the major surface and in fluid communication with the inlet. Each cell culture subunit also includes a cell culture substrate disposed on the major surface of the distribution plate. The system further includes a plurality of input lines for supplying at least one of cells, cell culture media, nutrients, and reagents to the plurality of cell culture subunits, each input line of the plurality of input lines being fluidly connected to the inlet. The plurality of outlets is configured to distribute at least one of cells, cell culture media, nutrients, and reagents from the plurality of input lines substantially uniformly across the cell culture substrate.

Additional aspect of embodiments of the present disclosure are described below. These aspects include the fixed-bed bioreactor system further including a vessel with an interior cavity arranged to house the plurality of cell culture subunits. The plurality cell culture subunits are modular and individually addable and/or removable from the vessel. The vessel is able to house a variable number of cell culture subunits. The cell culture substrate in each subunit has a height h that is less than or equal to a predetermined height, where the predetermined height is about 100 mm, 50 mm, 40 mm, 30 mm, 20 mm, or 10 mm.

Aspects of embodiments of the distribution plate include the plurality of outlets being arrayed across a diameter of the major surface. The distribution plate of a first cell culture subunit of the plurality of cell culture subunits has a central plate bore sized to allow an input line of a second cell culture subunit of the plurality of cell culture subunits to pass through the first cell culture subunit. The cell culture substrate can also include a central substrate bore coaxially aligned with the central plate bore. The inlet for the distribution plate can be disposed radially outward from the central plate bore. Thus, at least one of the plurality of input lines is curved or bent such that the input line can pass through a central plate bore of a first cell culture subunit and then extend radially outward to the inlet of a second cell culture subunit. In addition, the cell culture substrate can have at least one cored section to increase permeability of fluid throughout the cell culture substrate.

Aspects of the substrate in some embodiments include the cell culture substrate being a dissolvable foam scaffold. The dissolvable foam scaffold can include an ionotropically crosslinked polygalacturonic acid compound selected from at least one of: pectic acid; partially esterified pectic acid, partially amidated pectic acid and salts thereof. The dissolvable foam scaffold can further include at least one first water-soluble polymer having surface activity

Additional aspects of the present disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 shows an example of a bioreactor system having a unitary fixed bed substrate.

FIG. 2 shows a bioreactor system having a modular fixed bed substrate arrangement, according to one or more embodiments of this disclosure.

FIG. 3 is a cross-section of a bioreactor system having a modular fixed bed substrate arrangement, according to one or more embodiments of this disclosure.

FIG. 4 is a close-up cross-section view of one of the modular units from FIG. 3, according to one or more embodiments of this disclosure.

FIG. 5 shows a modular fixed-bed bioreactor system, according to one or more embodiments of this disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure relate to fixed-bed bioreactor systems with modular designs and improved fluid flow and diffusion characteristics in the packed-bed cell culture substrate. In conventional large-scale cell culture bioreactors, different types of fixed-bed bioreactors have been used. Usually these fixed beds contain porous matrices to retain adherent or suspension cells, and to support growth and proliferation. Fixed-bed matrices provide high surface area to volume ratios, so cell density can be higher than in the other systems. However, the fixed bed often functions as a depth filter, where cells are physically trapped or entangled in fibers of the matrix. Thus, because of linear flow of the cell inoculum through the fixed bed, cells are subject to heterogeneous distribution inside the fixed-bed, leading to variations in cell density through the depth or width of the fixed bed. For example, cell density may be higher at the inlet region of a bioreactor and significantly lower nearer to the outlet of the bioreactor. This non-uniform distribution of the cells inside of the fixed bed significantly hinders scalability and predictability of such bioreactors in bioprocess manufacturing, and can even lead to reduced efficiency in terms of growth of cells or viral vector production per unit surface area or volume of the fixed bed.

Another problem encountered in fixed-bed bioreactors disclosed in prior art is the channeling effect. Due to random nature of packed nonwoven fibers, the local fiber density at any given cross section of the fixed bed is not uniform. Medium flows quickly in the regions with low fiber density (high bed permeability) and much slower in the regions of high fiber density (lower bed permeability). The resulting non-uniform media perfusion across the fixed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to fixed-bed functions as depth filter, cells that are released at the end of cell culture process are entrapped inside the fixed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

The above limitations of conventional bioreactors and/or fixed-bed substrates can lead to diffusional limitations with respect to the cell nutrients contained in cell culture media that is perfused through the bioreactor. The dimensions of the packed bed can be a factor in this. For example, a fixed bed of a certain size might not be able to deliver nutrients to cells in the downstream sections of the fixed bed. For this reason, one or more embodiments of this disclosure include modular cell culture subunits having fixed bed substrates of a predetermined or limited size. This predetermined size can be designed to allow nutrient perfusion throughout the substrate that is sufficient for the given cell culture application. In addition, modifications to the cell culture substrate (e.g., by include cores or channels within the cell culture substrate) can help distribute media or fluid evenly through the substrate.

The embodiments disclosed herein enable efficient and high-yield cell culturing for anchorage-dependent cells and production of cell products (e.g., proteins, antibodies, viral particles). Embodiments include a porous cell-culture matrix made from porous substrates (such as a dissolvable foam scaffold or an ordered and regular array of porous substrate material such as mesh) that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. By using cell culture subunits that can be added to a reactor vessel in varying quantities, the cell culture surface area can be scaled as needed. For example, in some embodiments, a bioreactor can be easily scaled from process development scale to product scale with comparable viral genome per unit surface area of substrate (VG/cm 2) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce about 10¹⁶ to 10¹⁸ viral genomes (VG) per batch.

The present disclosure describes a modular fixed-bed bioreactor system having a plurality of cell culture subunits. Embodiments include the individual subunits of fixed-bed bioreactors, as well as the assembled plurality of subunits in a bioreactor system. Using individual cell culture subunits, each with its own fixed bed cell culture substrate, that can be combined together provides a solution that is scalable and removes the limitations of operational conditions imposed by nutrient and/or oxygen gradients within the packed bed during cell culture. Each individual subunits provides a short media perfusion path and thus supports optimal cell culture conditions. Multiple individual subunits can be assembled into one unit or vessel, thus providing scale up flexibility of the manufacturing process. Depending on the targeted yield of the production batch, an end user can configure a system to use any number of subunits, such as from 1 to 10 or more individual subunits simultaneously, for example.

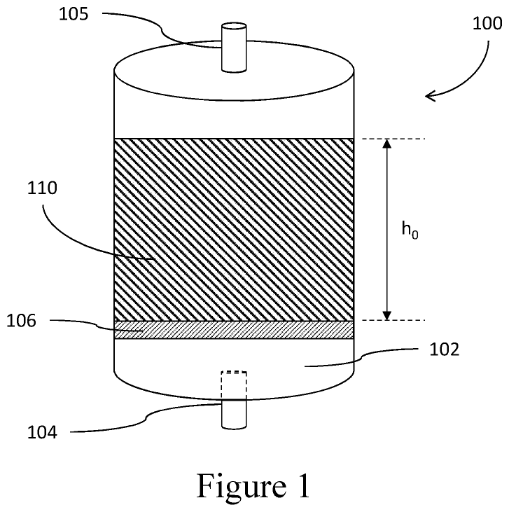

Referring to FIG. 1, a fixed-bed bioreactor 100 is shown. The fixed-bed bioreactor 100 includes an internal cavity 102 housing a cell culture substrate 110 disposed on a distribution plate 106. The internal cavity 102 is supplied with cells, cell culture media, or other fluid or nutrients via the input line 104. The media or other fluid from the input line 104 passes through the distribution plate 106 and is thus distributed across a portion of the cell culture substrate 110. After perfusing through the substrate 110, the extra fluid, along with any waste, cells, or cell by products, can be removed from the internal cavity 106 via the vessel outlet 105. As shown in FIG. 1, the cell culture substrate 110 has a height h₀. Due the relatively tall height h₀, it is possible that media and cell nutrients will not be efficiently supplied to the top portion (as viewed in FIG. 1) of the substrate 110. Therefore, embodiments discussed herein provide for cell culture subunits that each have lower heights and thus decreased chances of diffusional limitations.

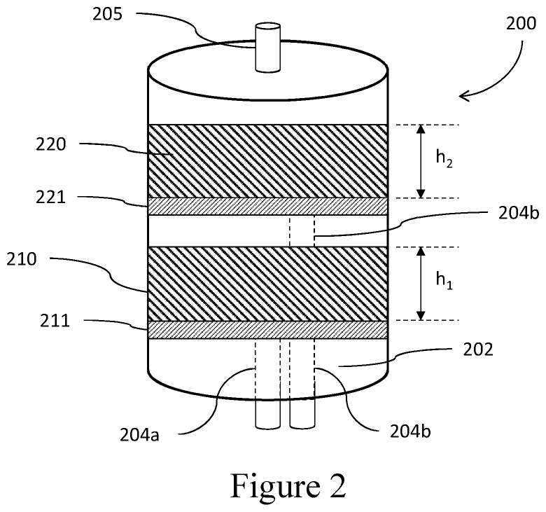

For example, as shown in FIG. 2, a fixed-bed bioreactor system 200 is shown according to one or more embodiments. The constructions of the fixed-bed bioreactor system 200 again includes an internal cavity 202 and a vessel outlet 205. However, in this embodiments, two cell culture subunits replace the single substrate of FIG. 1. The first subunit includes a distribution plate 211 and cell culture substrate 210 disposed thereon. The distribution plate 211 is fed with fluid via the input line 204a. The fixed-bed bioreactor system of FIG. 2 also shows a second cell culture subunit having distribution plate 221 and substrate 220 disposed thereon. The second subunit is supplied with a second input line 204b. When each subunit is similarly constructed in terms of the substrate and distribution plate, and each subunit has its own input line, the performance across the subunits can be kept consistent and predictable. Due to the modularity, subunits can be added or removed as needed with predictable results. And by improving on the fluid distribution through the substrate of each subunit, use of the available surface area of substrate material is maximized. Although not necessarily drawn to scale, it should be appreciated that the height (h₁, h₂) of the substrate in each subunit are is less than the height h₀ shown in FIG. 1. The predetermined height of each subunit can be less than or equal to about 500 mm, 200 mm, 100 mm, 50 mm, 40 mm, 30 mm, 20 mm, or 10 mm.

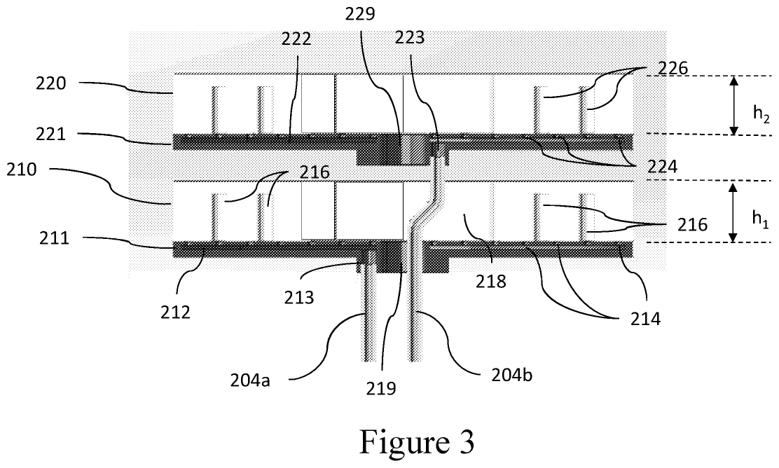

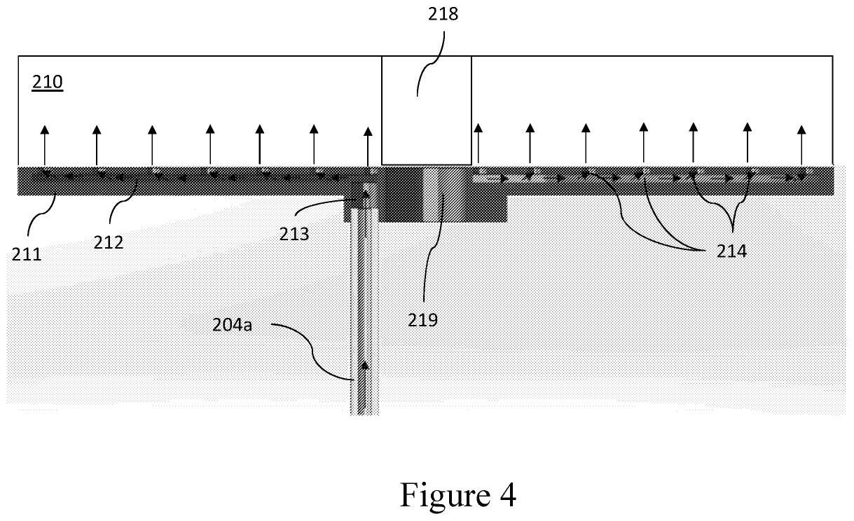

FIG. 3 shows an alternate cross-section view of a modular bioreactor system, similar to that shown in FIG. 2. The detailed view of FIG. 3 shows a close-up of the distribution plates 211, 221 and substrates 210, 220. Each distribution plate 211, 221 has an inlet 213, 223 that is connected to a respective input line 204a, 204b, respectively. These inlets 213, 223 are fluidly connected to a number of outlets 214, 224 on a major surface of the distribution plate; the major surface being that on which the cell culture substrate is disposed. This flow arrangement is shown in the close-up cross-section view of FIG. 4, where the straight arrows represent fluid flow from the input line 204a, into the inlet 213, through a radial flow path 212 of the distribution plate 211, and then up through the outlets 214 in the major surface of the distribution plate 211. In this way, fluid supplied by the input lines 204a, 204b can be distributed evenly across a bottom surface of the substrate. By distributing fluid in this way, while keeping the height h₁, h₂ of the substrates below a predetermined level, diffusion of nutrients to the extremities of the substrate can be improved. However, depending on the application and scale of the modular subunits, there may be an additional need for improved perfusion throughout the cell culture substrate 210, 220. Accordingly, as shown in FIG. 3, a number of cores 216, 226 can be removed from the substrates 210, 220 to open up additional fluid flow pathways in the substrates, thereby improving distribution of flow of media in the cell culture substrates. These cores 216, 226 are defined as sections of voids in the cell culture substrates that are removed from the substrate or are pre-formed into the substrate itself. These cores 216, 226 extend at least 20%, 25%, 50%, or 75% into the cell culture substrate in a thickness or height direction of the substrate, and such voids should not be confused with the cell culture substrate pores, which are on a much smaller scale.

To enable the modular, stackable arrangement of the cell culture subunits, as shown in FIG. 3, the input line 204b of the second subunit passes through the first subunit. In the embodiment shown in FIG. 3, the distribution plate 211 is provided with a central plate bore 219 to allow for passage of the input line 204b. Likewise, the cell culture substrate of the first subunit is provided with a central substrate bore, which can be coaxially aligned with the central plate bore, so that the input line can be passed through the cell culture substrate when stacking the modular subunits. Although the embodiments shown uses central bores through both the distribution plate 211 and cell culture substrate 210, it should be understood that the bores 219, 218 do not need to be centrally located and can be offset to any side, so long as the input line 204b can be fed to the inlet 223 of the second subunit. Similarly, the distribution plate 221 of the second subunit has a central plate bore 229 and the cell culture substrate 220 of the second subunit has a central substrate bore so that the input lines of one or more additional subunits can be passed therethrough to further expand the system.

The inlets 213, 223 of the distribution plates 211, 221 can be offset from the central plate bores 219, 229. In such as case, the input lines can be cured, kinked, offset, or flexible in a manner that allows the input line to pass through a central plate bore of one subunit while being able to be routed to the offset inlet, as shown for the input line 204b and inlet 223 in FIG. 3.

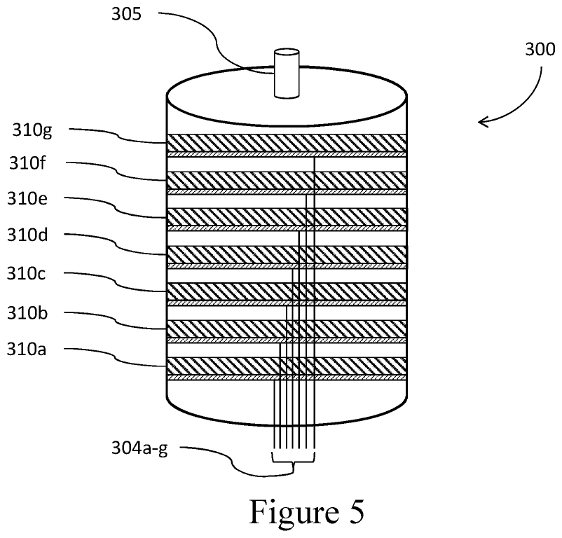

While the embodiments in FIGS. 2 and 3 show only two subunits, it is contemplated that embodiments of this disclosure can include more subunits with additional cell culture substrates. For example, FIG. 5 shows an embodiment of a modular bioreactor system 300 with seven subunits 310a-310g, each provided with a separate input line 304a-g. Seven is an example only, and the number of subunits in any one bioreactor vessel can be any number required.

According to one or more embodiments, after fluid (e.g., cell culture media) is passed through the cell culture substrate of a subunit, it then proceeds to flow out through an outlet (e.g., outlet 205 in FIG. 2 and outlet 305 in FIG. 5), after which point it can be recirculated and/or reconditioned, or otherwise processed. However, in some embodiments, each subunit may be directly connected to an outlet line of its own, and these outlet lines may all exit the vessel separately or be combined near an outlet 205, 305 before exiting the vessel.

In some embodiments, a dissolvable foam scaffold is used for the substrate. The foam scaffold is porous and enables excellent perfusion. For harvesting, the dissolvable foam scaffold can be dissolved or digested, efficiently releasing the cells and/or other cell culture products. In one embodiment, a substrate is provided with a structurally defined surface area for adherent cells to attach and proliferate that has good mechanical strength and forms a highly uniform multiplicity of interconnected fluidic networks when assembled in a fixed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a substrate is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed substrates, and can avoid formation of large and/or uncontrollable 3D cellular aggregates with limited nutrient diffusion and increased metabolite concentrations. Thus, the substrate eliminates diffusional limitations during operation of the bioreactor. In addition, the substrate enables easy and efficient cell harvest from the bioreactor.

In some embodiments of this disclosure, the cell culture substrates are dissolvable foam scaffolds for cell culture. The dissolvable foam scaffold is a porous foam that includes an open pore architecture. The dissolvable foam scaffold can have a porosity of from about 85% to about 96% and an average pore size diameter of between about 50 μm and about 500 μm. The dissolvable foam scaffold provides a protected environment within the pores of the foam scaffold for the culturing of cells. Additionally, the dissolvable foam scaffold is also dissolvable when exposed to an appropriate enzyme that digests or breakdowns the material which facilitates harvesting of the cells cultured in the scaffold without damaging the cells.

Dissolvable foam scaffolds as described herein include at least one ionotropically crosslinked polysaccharide. Generally, polysaccharides possess attributes beneficial to cell culture applications. Polysaccharides are hydrophillic, non-cytotoxic and stable in culture medium. Examples include pectic acid, also known as polygalacturonic acid (PGA), or salts thereof, partly esterified pectic acid or salts thereof, or partly amidated pectic acid or salts thereof. Pectic acid can be formed via hydrolysis of certain pectin esters. Pectins are cell wall polysaccharides and in nature have a structural role in plants. Major sources of pectin include citrus peel (e.g., peels from lemons and limes) and apple peel. Pectins are predominantly linear polymers based on a 1,4-linked alpha-D-galacturonate backbone, interrupted randomly by 1,2-linked L-rhamnose. The average molecular weight ranges from about 50,000 to about 200,000 Daltons.

The polygalacturonic acid chain of pectin may be partly esterified, e.g., methyl groups and the free acid groups may be partly or fully neutralized with monovalent ions such as sodium, potassium, or ammonium ions. Polygalacturonic acids partly esterified with methanol are called pectinic acids, and salts thereof are called pectinates. The degree of methylation (DM) for high methoxyl (HM) pectins can be, for example, from 60 to 75 mol % and those for low methoxyl (LM) pectins can be from 1 to 40 mol %. The degree of esterification of partly esterified polygalacturonic acids as described herein may be less than about 70 mol %, or less than about 60 mol %, or less than 50 mol %, or even less than about 40 mol %, and all values therebetween. Without wishing to be bound by any particular theory, it is believed that a minimum amount of free carboxylic acid groups (not esterified) facilitates a degree of ionotropic crosslinking which allow for the formation of a dissolvable scaffold which is insoluble.

Alternatively, the polygalacturonic acid chain of pectin may be partly amidated.

Polygalacturonic acids partly amidated pectin may be produced, for example, by treatment with ammonia. Amidated pectin contains carboxyl groups (˜COOH), methyl ester groups (˜COOCH₃), and amidated groups (—CONH₂). The degree of amidation may vary and may be, for example, from about 10% to about 40% amidated.

According to embodiments of the present disclosure, dissolvable foam scaffolds as described herein may include a mixture of pectic acid and partly esterified pectic acid. Blends with compatible polymers may also be used. For example, pectic acid and/or partly esterified pectic acid may be mixed with other polysaccharides such as dextran, substituted cellulose derivatives, alginic acid, starches, glycogen, arabinoxylans, agarose, etc. Glycosaminoglycans like hyaluronic acid and chondroitin sulfate, or various proteins such as elastin, fibrin, silk fibroin, collagen and their derivatives can be also used. Water soluble synthetic polymers can be also blended with pectic acid and/or partly esterified pectic acid. Exemplary water soluble synthetic polymers include, but are not limited to, polyalkylene glycol, poly(hydroxyalkyl(meth)acrylates), poly(meth)acrylamide and derivatives, poly(N-vinyl-2-pyrrolidone), and polyvinyl alcohol.

According to embodiments of the present disclosure, dissolvable foam scaffolds as described herein may further include at least one first polymer. The at least one first polymer is water soluble, non-ionotropically crosslinkable and has surface activity. As used herein, the term “surface activity” refers to the activity of an agent to lower or eliminate the surface tension (or interfacial tension) between two liquids or between a liquid and a solid or between gas and liquid. The at least one first polymer may have a hydrophilic-lipophilic balance (HLB) of greater than about 8 or even greater than about 10. For example, the at least one first polymer may have an HLB of between about 8 and about 40 or between about 10 and about 40. The at least one first polymer may have an HLB of between about 8 and about 15, or even between about 10 and about 12. HLB provides a reference for the lipophilic or hydrophilic degree of a polymer. A larger HLB value indicates stronger hydrophilicity, while a smaller HLB value indicates a stronger lipophilicity. In general, the HLB value varies in the range of from 1 to 40 and the hydrophilic-lipophilic transition is often considered to be between about 8 and about 10. When the HLB value is less than the hydrophilic-lipophilic transition, the material is lipophilic, and when the HLB value is greater than the hydrophilic-lipophilic transition the material is hydrophilic.

Exemplary first polymers in accordance with embodiments of the present disclosure may be any of cellulose derivatives, proteins, synthetic amphiphilic polymers, and combinations thereof. Exemplary cellulose derivatives include, but are not limited to, hydroxyethylcellulose (HEC), hydroxypropylcellulose (HPC), methylcellulose (MC), hydroxyethylmethylcellulose (HEMC), and hydroxypropyl-methylcellulose (HPMC). Exemplary proteins include, but are not limited to, bovine serum albumin (BSA), gelatine, casein and hydrophobins. Exemplary synthetic amphiphilic polymers include, but are not limited to, a poloxamer available under the trade name Synperonics® (commercially available from Croda International, Snaith, United Kingdom), a poloxamer available under the trade name Pluronics® (commercially available from BASF Corp., Parsippany, NJ) and a poloxamer available under the trade name Kolliphor® (commercially available from BASF Corp., Parsippany, NJ).

Dissolvable foam scaffolds as described herein may further include at least one second polymer. The at least one second polymer is water soluble and has no surface activity. Exemplary second polymers may be any of synthetic polymers, semisynthetic polymers, natural polymers and combinations thereof. Exemplary synthetic polymers include, but are not limited to, polyethylene glycol, polyvinyl pyrrolidone, polyvinyl alcohol, carboxyvinyl polymer, polyacrylic acid, polyacrylamide, homopolymer and copolymer of N-(2-Hydroxypropyl) methacrylamide, polyvinyl methyl ether-maleic anhydride, and polyethylene oxide/polypropylene oxide block copolymers. Exemplary semisynthetic polymers include, but are not limited to, dextran derivatives, carboxymethyl cellulose, hydroxyethyl cellulose and derivatives, methylcellulose and derivatives, ethylcellulose cellulose, ethyl hydroxyethyl cellulose, and hydroxypropyl cellulose. Exemplary natural polymers include, but are not limited to, starch and starch derivatives, polymers obtained by microbial fermentation such as curdlan, pullulan and gellan gum, xanthan gum, dextran, proteins such as albumin, casein and caseinates, gelatin, seaweed extracts such as agar, alginates and carrageenan, seed extracts such as guar gum and derivatives and locust bean gum, hyaluronic acid, and chondroitin sulfate.

Dissolvable foam scaffolds as described herein may be crosslinked to increase their mechanical strength and to prevent the dissolution of the scaffolds when placed in contact with cell culture medium. Crosslinking may be performed by ionotropic gelation as described below wherein ionotropic gelation is based on the ability of polyelectrolytes to crosslink in the presence of multivalent counter ions to form crosslinked scaffolds. Without wishing to be bound by any particular theory, it is believed that ionotropic gelation of the polysaccharide of the dissolvable foam scaffolds is the result of strong interactions between divalent cations and the polysaccharide.

According to embodiments of the present disclosure, scaffolds as described herein are porous foam scaffolds. Foam scaffolds as described herein may have a porosity of from about 85% to about 96%. For example, foam scaffolds as described herein may have a porosity of from about 91% to about 95%, or about 94% to about 96%. As used herein, the term “porosity” refers to the measure of open pore volume in the dissolvable scaffold and is referred to in terms of % porosity, wherein % porosity is the percent of voids in the total volume of the dissolvable foam scaffold. Foam scaffolds as described herein may have an average pore size diameter of between about 50 μm and about 500 For example, average pore size diameter may be between about μm and about 450 or between about 100 μm and about 400 or even between 150 and about 350 μm and all values therebetween.

Scaffolds as described herein may have a wet density of less than about 0.40 g/cc. For example, scaffolds as described herein may have a wet density of less than about 0.35 g/cc, or less than about 0.30 g/cc, or less than about 0.25 g/cc. Scaffolds as described herein may have a wet density of between about 0.16 g/cc and about 0.40 g/cc, or between about 0.16 g/cc and about 0.35 g/cc, or between about 0.16 g/cc and about 0.30 g/cc, or even between about 0.16 g/cc and about 0.25 g/cc, and all values therebetween. Scaffolds as described herein may have a dry density of less than about 0.20 g/cc. For example, scaffolds as described herein may have a dry density of less than about 0.15 g/cc, or less than about 0.10 g/cc, or less than about 0.05 g/cc. Scaffolds as described herein may have a dry density of between about 0.02 g/cc and about 0.20 g/cc, or between about 0.02 g/cc and about 0.15 g/cc, or between about 0.02 g/cc and about 0.10 g/cc, or even between about 0.02 g/cc and about 0.05 g/cc, and all values therebetween.

Several pore types are possible in scaffolds. Open pores allow for cellular access on both sides of the scaffold and allow for liquid flow and transport of nutrients through the dissolvable scaffold. Partially open pores allow for cellular access on one side of the scaffold, but mass transport of nutrients and waste products is limited to diffusion. Closed pores have no openings and are not accessible by cells or by mass transport of nutrients and waste products. Dissolvable foam scaffolds as described herein have an open pore architecture and highly interconnected pores. Generally, the open pore architecture and highly interconnected pores enable migration of cells into the pores of the dissolvable foam scaffolds and also facilitate enhanced mass transport of nutrients, oxygen and waste products. The open pore architecture also influences cell adhesion and cell migration by providing a high surface area for cell-to-cell interactions and space for ECM regeneration.

Dissolvable foam scaffolds as described herein are digested when exposed to an appropriate enzyme that digests or breakdowns the material. Non-proteolytic enzymes suitable for digesting the foam scaffolds, harvesting cells, or both, include pectinolytic enzymes or pectinases, which are a heterogeneous group of related enzymes that hydrolyze the pectic substances. Pectinases (polygalacturonase) are enzymes that break down complex pectin molecules to shorter molecules of galacturonic acid. Commercially available sources of pectinases are generally multi-enzymatic, such as Pectinex™ ULTRA SP-L (commercially available from Novozyme North American, Inc., Franklinton, NC), a pectolytic enzyme preparation produced from a selected strain of Aspergillus aculeatus. Pectinex™ ULTRA SP-L contains mainly polygalacturonase, (EC 3.2.1.15) pectintranseliminase (EC 4.2.2.2) and pectinesterase (EC: 3.1.1.11). The EC designation is the Enzyme Commission classification scheme for enzymes based on the chemical reactions the enzymes catalyze.

According to embodiments of the present disclosure, digestion of the dissolvable foam scaffolds also includes exposing the scaffold to a divalent cation chelating agent. Exemplary chelating agents include, but are not limited to, ethylenediaminetetraacetic acid (EDTA), cyclohexanediaminetetraacetic (CDTA), ethylene glycol tetraacetic acid (EGTA), citric acid and tartaric acid.

The time to complete digestion of dissolvable foam scaffolds as described herein may be less than about 1 hour. For example, the time to complete digestion of foam scaffolds may be less than about 45 minutes, or less than about 30 minutes, or less than about 15 minutes, or between about 1 minute and about 25 minutes, or between about 3 minutes and about 20 minutes, or even between about 5 minutes and about 15 minutes.

According to embodiments of the present disclosure, scaffolds as described herein may further include an adhesion polymer coating. The adhesion polymer may include peptides. Exemplary peptides may include, but are not limited to BSP, vitronectin, fibronectin, laminin, Type I and IV collagen, denatured collagen (gelatin), and like peptides, and mixtures thereof. Additionally, the peptides may be those having an RGD sequence. The coating may be, for example, Synthemax® II-SC (commercially available from Corning, Incorporated, Corning, NY). Optionally, the adhesion polymer may include an extracellular matrix. The coating may be, for example, Matrigel® (commercially available from Corning, Incorporated, Corning, NY).

Additional details and examples of dissolvable foam scaffolds contemplated in embodiments of this disclosure are described in U.S. patent application Ser. No. 16/765,722, the content of which is incorporated herein by reference.

In one or more additional embodiments of this disclosure include a cell culture substrate having a defined and ordered structure, in contrast to existing cell culture substrates used in cell culture bioreactors (i.e., non-woven substrates of randomly ordered fibers). The defined and order structure allows for consistent and predictable cell culture results. In addition, the substrate has an open porous structure that prevents cell entrapment and enables uniform flow through the fixed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest. Examples of embodiments of structurally defined or woven substrates are described in U.S. patent application Ser. No. 16/781,685, the contents of which are incorporated herein by reference.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, production is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

By using a structurally defined culture matrix of sufficient rigidity, high-flow-resistance uniformity across the matrix or fixed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the fixed bed configuration, allowing for complete cell harvest with high viability at the end of culturing. The matrix also delivers packaging uniformity for the fixed bed, and enables direct scalability from process development units to large-scale industrial bioprocessing unit. The ability to directly harvest cells from the fixed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

Substrates for adherent cells in existing bioreactors do not exhibit this behavior and instead their fixed beds tend to create preferential flow channels and have substrate materials with anisotropic permeability. The flexibility of the cell culture substrates of the current disclosure allows for their use in various applications and bioreactor or container designs while enabling better and more uniform permeability throughout the bioreactor vessel.

As discussed herein, the cell culture substrate can be used within a bioreactor vessel, according to one or more embodiments. For example, the substrate can be used in a fixed-bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

Embodiments of this disclosure include cell culture systems that also include one or more sensors, a user interface and controls, and various inlet and outlets for media and cells. According to some embodiments, a media conditioning vessel is controlled by a controller to provide the proper temperature, pH, O₂, and nutrients for the cell culture application at any given time. While in some embodiments, the bioreactor can also be controlled by the controller, in other embodiments the bioreactor is provided in a separate perfusion circuit, where a pump is used to control the flow rate of media through the perfusion circuit based on the detection of O2 at or near the outlet of the bioreactor.

The embodiments of cell culture systems disclosed herein can be used in methods of cell culture involving process steps that can include seeding and attaching cells to the cell culture substrate, expanding the seeded and/or attached cells during a period of cell expansion, transfecting the cells for viral vector production applications, producing viral vector, and harvesting the cells, virus, or other components.

During these steps of the methods, the values of pH₁, pO₁, [glucose]₁, pH₂, pO₂, [glucose]₂, and maximum flow rate can be measured to monitor the state of the cell culture. For example, the values for pH₁, pO₁, and glucose₁ can be measured within the cell culture chamber of the bioreactor system, and pH₂, pO₂, and glucose₂ can be measured by sensors at the outlet of the bioreactor vessel. Based on these values, a perfusion pump control unit makes determinations to maintain or adjust the perfusion flow rate. For example, a perfusion flow rate of the cell culture media to the cell culture chamber may be continued at a present rate if at least one of pH₂≥pH₂ₘᵢₙ, pO₂≥pO₂ₘᵢₙ, and [glucose]₂≥[glucose]₂ₘᵢₙ. If the current flow rate is less than or equal to a predetermined max flow rate of the cell culture system, the perfusion flow rate is increased. Further, if the current flow rate is not less than or equal to the predetermined max flow rate of the cell culture system, a controller of the cell culture system can reevaluate at least one of: (1) pH₂ₘᵢₙ, pO₂ₘᵢₙ, and [glucose]₂ₘᵢₙ; (2) pH₁, pO₁, and [glucose]₁; and (3) a height of the bioreactor vessel.

The cell culture substrate can be arranged in multiple configurations within the culture chamber depending on the desired system. For example, in one or more embodiments, the system includes one or more layers of the substrate with a width extending across the width of an interior cavity of a cell culture vessel. Multiple layers of the substrate may be stacked in this way to a predetermined height. As discussed above, the substrate layers may be arranged such that the first and second sides of one or more layers are perpendicular to a bulk flow direction of culture media through the interior cavity, or the first and second sides of one or more layers may be parallel to the bulk flow direction. In one or more embodiments, the cell culture substrate includes one or more layers at a first orientation with respect to the bulk flow, and one or more other layers at a second orientation that is different from the first orientation. For example, various layers may have first and second sides that are parallel or perpendicular to the bulk flow direction, or at some angle in between. In some embodiments, the cell culture substrate is a monolithic porous substrate, such as a foam scaffold. Each cell culture subunit can contain a single foam scaffold, according to some preferred embodiments. However, in one or more embodiments, each cell culture subunit can contain multiple dissolvable foam scaffolds. In the case of multiple dissolvable foam scaffold per subunit, the foam scaffolds can be arranged in a plurality of layer (e.g., a stack of foam disks) or can be a packed-bed of small strips, chunks, or beads of dissolvable foam scaffold. However, in some applications, it may be possible to have better control of fluid flow and diffusion through a monolithic foam scaffold with a defined structure, as opposed to a plurality of smaller pieces packed together, which can result in uneven flow characteristics through the packed bed.

In one or more embodiments, the cell culture system includes a plurality of discrete pieces of the cell culture substrate in a fixed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The fixed bed cell culture matrix of one or more embodiments can consist of the woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

The embodiments disclosed herein have advantages over the existing platforms for cell culture and viral vector production. It is noted that the embodiments of this disclosure can be used for the production of a number of types of cells and cell byproducts, including, for example, adherent or semi-adherent cells, Human embryonic kidney (HEK) cells (such as HEK23), including transfected cells, viral vectors, such as Lentivirus (stem cells, CAR-T) and Adeno-associated virus (AAV). These are examples of some common applications for a bioreactor or cell culture substrate as disclosed herein, but are not intended to be limiting on the use or applications of the disclosed embodiments, as a person of ordinary skill in the art would understand the applicability of the embodiments to other uses.

As discussed herein, the embodiments of this disclosure provide cell culture substrates, bioreactor systems, and methods of culturing cells or cell by-products that are scalable and can be used to provide a cell seed train to gradually increase a cell population. One problem in existing cell culture solutions is the inability for a given bioreactor system technology to be part of a seed train. Instead, cell populations are usually scaled up on various cell culture substrates. This can negatively impact the cell population, as it is believed that cells become acclimated to certain surfaces and being transferred to a different type of surface can lead to inefficiencies. Thus, it would be desirable to minimize such transitions between cell culture substrates or technologies. By using the same cell culture substrate across the seed train, as enabled by embodiments of this disclosure, efficiency of scaling up a cell population is increased. For example, the seed train can begin with a vial of starter cells which are seeded into a first vessel having one or more cell culture subunits of a predetermined three-dimensional cell culture surface area (e.g., a predetermined thickness, width, and/or porosity). After culturing cells for a time in the first vessel, the cells can be harvested and fully or partially reseeded into a second vessel having a higher number of cell culture subunits and/or subunits of a greater cell culture surface area, so that the population of cells can be expanded. This process of harvest and reseeding to expand the culture can be repeated as desired. At the end of this seed train, the cells can be seeded into a production-scale bioreactor vessel according to embodiments of this disclosure, with a surface area of about 5,000,000 cm², for example. Harvest and purification steps can then be performed when the cell culture is complete. Harvest can be accomplished via digestion of the dissolvable cell culture substrate, or by in situ cell lysis with a detergent (such as Triton X-100), or via mechanical lysis; and further downstream processing can be performed, as needed.

The benefits of using the same cell culture substrate within the seed train (e.g., from process development level to pilot level, or even to production level) include efficiencies gained from the cells being accustomed to the same surface during the seed train and production stages; a reduced number of manual, open manipulations during seed train phases; more efficient use of the fixed bed due to uniform cell distribution and fluid flow, as described herein; and the flexibility of using mechanical or chemical lysis during viral vector harvest.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a fixed-bed bioreactor system for culturing cells, the system comprising: a plurality of cell culture subunits, each cell culture subunit comprising: a distribution plate comprising a major surface configured to support a cell culture substrate, an inlet, and a plurality of outlets disposed on the major surface and in fluid communication with the inlet; and a cell culture substrate disposed on the major surface of the distribution plate. The system also comprising a plurality of input lines configured for supplying at least one of cells, cell culture media, nutrients, and reagents to the plurality of cell culture subunits, each input line of the plurality of input lines being fluidly connected to the inlet, wherein the plurality of outlets is configured to distribute at least one of cells, cell culture media, nutrients, and reagents from the plurality of input lines substantially uniformly across the cell culture substrate.

Aspect 2 pertains to the fixed-bed bioreactor system of Aspect 1, further comprising a vessel comprising an interior cavity configured to house the plurality of cell culture subunits.

Aspect 3 pertains to the fixed-bed bioreactor system of Aspect 2, wherein the plurality cell culture subunits are modular and individually addable and/or removable from the vessel.

Aspect 4 pertains to the fixed-bed bioreactor system of Aspect 2 or Aspect 3, wherein the vessel is configured to house a variable number of cell culture subunits.

Aspect 5 pertains to the fixed-bed bioreactor system of Aspect 1, wherein the cell culture substrate comprises a polymer.

Aspect 6 pertains to the fixed-bed bioreactor system of any one of Aspects 1-5, wherein the cell culture substrate comprises a height h that is less than or equal to a predetermined height.

Aspect 7 pertains to the fixed-bed bioreactor system of Aspect 6, wherein the predetermined height is about 100 mm, 50 mm, 40 mm, 30 mm, 20 mm, or 10 mm.

Aspect 8 pertains to the fixed-bed bioreactor system of any one of Aspects 1-7, wherein the plurality of outlets is arrayed across a diameter of the major surface.

Aspect 9 pertains to the fixed-bed bioreactor system of any one of Aspects 1-8, wherein the distribution plate of a first cell culture subunit of the plurality of cell culture subunits comprises a central plate bore sized to allow an input line of a second cell culture subunit of the plurality of cell culture subunits to pass through the first cell culture subunit.

Aspect 10 pertains to the fixed-bed bioreactor system of Aspect 9, wherein the cell culture substrate comprises a central substrate bore coaxially aligned with the central plate bore.

Aspect 11 pertains to the fixed-bed bioreactor system of Aspect 9 or Aspect 10, wherein the inlet is disposed radially outward from the central plate bore.

Aspect 12 pertains to the fixed-bed bioreactor system of Aspect 11, wherein at least one of the plurality of input lines is curved or bent such that the input line is configured to pass through a central plate bore of a first cell culture subunit and then extend radially outward to the inlet of a second cell culture subunit.

Aspect 13 pertains to the fixed-bed bioreactor system of any one of Aspects 1-12, wherein the cell culture substrate comprises at least one cored section configured to increase permeability of fluid throughout the cell culture substrate.

Aspect 14 pertains to the fixed-bed bioreactor system of any one of Aspects 1-13, further comprising a media conditioning vessel supplying the plurality of input lines.

Aspect 15 pertains to the fixed-bed bioreactor system of any one of Aspects 1-14, further comprising a plurality of media conditioning vessels supplying the plurality of input lines.

Aspect 16 pertains to the fixed-bed bioreactor system of Aspect 1, wherein the cell culture substrate comprises a dissolvable foam scaffold.

Aspect 17 pertains to the fixed-bed bioreactor system of Aspect 16, wherein the dissolvable foam scaffold comprises an ionotropically crosslinked polygalacturonic acid compound selected from at least one of: pectic acid; partially esterified pectic acid, partially amidated pectic acid and salts thereof.

Aspect 18 pertains to the fixed-bed bioreactor system of Aspect 17, wherein the dissolvable foam scaffold further comprises at least one first water-soluble polymer having surface activity.

Aspect 19 pertains to the fixed-bed bioreactor system of Aspect 17 or Aspect 18, wherein the dissolvable foam scaffold further comprises a water soluble plasticizer.

Aspect 20 pertains to the fixed-bed bioreactor system of Aspect 19, the dissolvable foam scaffold comprising less than about 55 wt. % water soluble plasticizer.

Aspect 21 pertains to the fixed-bed bioreactor system of Aspect 20, the dissolvable foam scaffold comprising between about 15 wt. % and about 55 wt. % water soluble plasticizer.

Aspect 22 pertains to the fixed-bed bioreactor system of any one of Aspects 16-21, the dissolvable foam scaffold further comprising an adhesion polymer coating.

Aspect 23 pertains to the fixed-bed bioreactor system of Aspect 22, wherein the adhesion polymer coating comprises peptides.

Aspect 24 pertains to the fixed-bed bioreactor system of Aspect 22, wherein the adhesion polymer coating comprises peptides selected from the group consisting of BSP, vitronectin, fibronectin, laminin, Type I collagen, Type IV collagen, denatured collagen and mixtures thereof.

Aspect 25 pertains to the fixed-bed bioreactor system of Aspect 22, wherein the adhesion polymer coating comprises Synthemax® II-SC.

Aspect 26 pertains to the fixed-bed bioreactor system of any one of Aspects 16-25, wherein the dissolvable foam scaffold comprises an average pore size diameter of between about 50 μm and about 500 μm.

Aspect 27 pertains to the fixed-bed bioreactor system of any one of Aspects 16-26, wherein the dissolvable foam scaffold comprises a wet density of less than about 0.40 g/cc.

Aspect 28 pertains to the fixed-bed bioreactor system of any one of Aspects 16-27, wherein the dissolvable foam scaffold comprises an open pore architecture.

Aspect 29 pertains to the fixed-bed bioreactor system of any one of Aspects 16-28, wherein the dissolvable foam scaffold comprises a porosity of between about 85% and about 96%.

Aspect 30 pertains to the fixed-bed bioreactor system of any one of Aspects 1-15, wherein the cell culture substrate comprises a structurally defined porous material.

Aspect 31 pertains to the fixed-bed bioreactor system of Aspect 30, wherein the cell culture substrate comprises a plurality of layers of the structurally defined porous material.

Aspect 32 pertains to the fixed-bed bioreactor system of Aspect 30 or Aspect 31, wherein the cell culture substrate comprises at least one of polystyrene, polyethylene terephthalate, polycarbonate, polyvinylpyrrolidone, polybutadiene, polyvinylchloride, polyethylene oxide, polypyrroles, and polypropylene oxide.

Aspect 33 pertains to the fixed-bed bioreactor system of any one of Aspects 30-32, wherein the cell culture substrate comprises at least one of a molded polymer lattice, a 3D-printed polymer lattice sheet, and a woven mesh sheet.

Aspect 34 pertains to the fixed-bed bioreactor system of any one of the preceding Aspects, wherein the cell culture substrate comprises a substantially uniform porosity.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A fixed-bed bioreactor system for culturing cells, the system comprising:
a plurality of cell culture subunits, each cell culture subunit comprising:
a distribution plate comprising a major surface configured to support a cell culture substrate, an inlet, and a plurality of outlets disposed on the major surface and in fluid communication with the inlet; and
a cell culture substrate disposed on the major surface of the distribution plate; and
a plurality of input lines configured for supplying at least one of cells, cell culture media, nutrients, and reagents to the plurality of cell culture subunits, each input line of the plurality of input lines being fluidly connected to the inlet,
wherein the plurality of outlets is configured to distribute at least one of cells, cell culture media, nutrients, and reagents from the plurality of input lines substantially uniformly across the cell culture substrate.

2. The fixed-bed bioreactor system of claim 1, further comprising a vessel comprising an interior cavity configured to house the plurality of cell culture subunits.

3. The fixed-bed bioreactor system of claim 2, wherein the plurality cell culture subunits are modular and individually addable and/or removable from the vessel.

4. The fixed-bed bioreactor system of claim 2, wherein the vessel is configured to house a variable number of cell culture subunits.

5. The fixed-bed bioreactor system of claim 1, wherein the cell culture substrate comprises a polymer.

6. The fixed-bed bioreactor system of claim 1, wherein the cell culture substrate comprises a height h that is less than or equal to a predetermined height.

7. The fixed-bed bioreactor system of claim 6, wherein the predetermined height is about 100 mm, 50 mm, 40 mm, 30 mm, 20 mm, or 10 mm.

8. The fixed-bed bioreactor system of claim 1, wherein the plurality of outlets is arrayed across a diameter of the major surface.

9. The fixed-bed bioreactor system of claim 1, wherein the distribution plate of a first cell culture subunit of the plurality of cell culture subunits comprises a central plate bore sized to allow an input line of a second cell culture subunit of the plurality of cell culture subunits to pass through the first cell culture subunit.

10. The fixed-bed bioreactor system of claim 9, wherein the cell culture substrate comprises a central substrate bore coaxially aligned with the central plate bore.

11. The fixed-bed bioreactor system of claim 9, wherein the inlet is disposed radially outward from the central plate bore.

12. The fixed-bed bioreactor system of claim 11, wherein at least one of the plurality of input lines is curved or bent such that the input line is configured to pass through a central plate bore of a first cell culture subunit and then extend radially outward to the inlet of a second cell culture subunit.

13. The fixed-bed bioreactor system of claim 1, wherein the cell culture substrate comprises at least one cored section configured to increase permeability of fluid throughout the cell culture substrate.

14. The fixed-bed bioreactor system of claim 1, further comprising a media conditioning vessel supplying the plurality of input lines.

15. The fixed-bed bioreactor system of claim 1, further comprising a plurality of media conditioning vessels supplying the plurality of input lines.

16. The fixed-bed bioreactor system of claim 1, wherein the cell culture substrate comprises a dissolvable foam scaffold.

17-29. (canceled)

30. The fixed-bed bioreactor system of claim 1, wherein the cell culture substrate comprises a structurally defined porous material.

31. The fixed-bed bioreactor system of claim 30, wherein the cell culture substrate comprises a plurality of layers of the structurally defined porous material.

32. (canceled)

33. The fixed-bed bioreactor system of claim 30, wherein the cell culture substrate comprises at least one of a molded polymer lattice, a 3D-printed polymer lattice sheet, and a woven mesh sheet.

34. The fixed-bed bioreactor system of claim 1, wherein the cell culture substrate comprises a substantially uniform porosity.
